(19) United States
(12) Patent Application Publication
(10) Pub. No.: US 2012/0203294 A1
(43) Pub. Date: Aug. 9, 2012

(54) CERAMIC BUSHING HAVING HIGH CONDUCTIVITY CONDUCTING ELEMENTS

(75) Inventor: Jens Troetzschel, Neuwiedermus (DE)

(73) Assignee: HERAEUS PRECIOUS METALS GMBH & CO. KG, Hanau (DE)

(21) Appl. No.: 13/361,388

(22) Filed: Jan. 30, 2012

Related U.S. Application Data
(60) Provisional application No. 61/438,051, filed on Jan. 31, 2011.

(30) Foreign Application Priority Data
Jan. 31, 2011 (DE) 10 2011 009 863.1

(51) Int. Cl. A61N 1/39; A61N 1/362; H01B 17/26

(52) U.S. Cl. 607/5; 174/152 R; 607/9

(57) ABSTRACT

One aspect relates to an electrical bushing for use in a housing of an implantable medical device. The electrical bushing includes at least one electrically insulating base body and at least one electrical conducting element. The conducting element is set-up to establish, through the base body, at least one electrically conductive connection between an internal space of the housing and an external space. The conducting element is hermetically sealed with respect to the base body. The at least one conducting element includes at least one cermet.

The at least one conducting element has a cross-section, a length, and a resistivity which provide the electrically conductive connection to have an ohmic series resistance of less than or equal to 1 Ohm.

One aspect also relates to and implantable medical device and a use of at least one cermet-comprising conducting element in an electrical bushing for an implantable medical device.

### CROSS-REFERENCE TO RELATED APPLICATIONS

This Non-Provisional Patent Application claims the benefit of the filing date of U.S. Provisional Patent Application Ser. No. 61/438,051, filed Jan. 31, 2011, entitled “CERAMIC BUSHING HAVING HIGH CONDUCTIVITY CONDUCTING ELEMENTS,” and this Patent Application also claims priority to German Patent Application No. DE 10 2011 009 863.1, filed on Jan. 31, 2011, and both of which are incorporated herein by reference.

### BACKGROUND

One aspect relates to an electrical bushing for use in a housing of an implantable medical device. Moreover, one aspect relates to a method for the manufacture of an electrical bushing for an implantable medical device.

The post-published document, DE 10 2009 035 972, discloses an electrical bushing for an implantable medical device having the features of the preamble of claim 1. Moreover, a use of at least one cermet-comprising conducting element in an electrical bushing for an implantable medical device and a method for the manufacture of an electrical bushing for an implantable medical device are disclosed.

A multitude of electrical bushings for various applications are known, examples including: U.S. Pat. No. 4,678,868, U.S. Pat. No. 7,564,674 B2, US 2008/0119906 A1, U.S. Pat. No. 7,145,076 B2, U.S. Pat. No. 7,561,917, US 2007/0183118 A1, U.S. Pat. No. 7,260,434B1, U.S. Pat. No. 7,761,165, U.S. Pat. No. 7,742,817 B2, U.S. Pat. No. 7,736,191 B1, US 2006/0259093 A1, U.S. Pat. No. 7,274,963 B2, US 2004116976 A1, U.S. Pat. No. 7,794,256, US 2010/0023086 A1, U.S. Pat. No. 7,502,217 B2, U.S. Pat. No. 7,706,124 B2, U.S. Pat. No. 6,999,818 B2, EP 1754511 A2, U.S. Pat. No. 7,035,076, EP 1685874 A1, WO 03/073450 A1, U.S. Pat. No. 7,136,273, U.S. Pat. No. 7,765,005, WO 2008/103166 A1, US 2008/0269831, U.S. Pat. No. 7,174,219 B2, WO 2004/110555 A1, U.S. Pat. No. 7,720,538 B2, WO 2010/091435, US 2010/0258342 A1, US 2001/0013756 A1, U.S. Pat. No. 4,315,054, and EP 0877400.

DE 697 297 19 T2 describes an electrical bushing for an active implantable medical device—also called implantable device or therapeutic device. Electrical bushings of this type serve to establish an electrical connection between a hermetically sealed interior and an exterior of the therapeutic device. Known implantable therapeutic devices are cardiac pacemakers or defibrillators, which usually include a hermetically sealed metal housing which is provided with a connection body, also called header, on one of its sides. Said connection body includes a hollow space having at least one connection socket for connecting electrode leads. In this context, the connection socket includes electrical contacts in order to electrically connect the electrode leads to the control electronics on the interior of the housing of the implantable therapeutic device. Hermetic sealing with respect to a surrounding is an essential prerequisite of an electrical bushing of this type. Therefore, lead wires that are introduced into an electrically insulating base body—also called signal-transmission elements—through which the electrical signals are propagated, must be introduced into the base body such as to be free of gaps. In this context, it has proven to be challenging that the lead wires generally are made of a metal and are introduced into a ceramic base body. In order to ensure a durable connection between the two elements, the internal surface of a through-opening—also called openings—in the base body is metallized for attachment of the lead wires by soldering. However, the metallization in the through-opening has proven to be difficult to apply. Only expensive procedures ensure homogeneous metallization of the internal surface of the bore hole and thus a hermetically sealed connection of the lead wires to the base body by soldering. The soldering process itself requires additional components, such as solder rings. Moreover, the process of connecting the lead wires to the previously metallized insulators utilizing the solder rings is a process that is laborious and difficult to automate. For example, the prior art does not provide a way of manufacturing, with simplified means, electrical bushings which feature highly tight sealing and good electrical properties simultaneously.

For these and other reasons there is a need for the present invention.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Further measures and advantages of the invention are evident from the claims, the description provided hereinafter, and the drawings. The invention is illustrated through several exemplary embodiments in the drawings. In this context, equal or functionally equal or functionally corresponding elements are identified through the same reference numbers. The invention shall not be limited to the exemplary embodiments.

FIG. 1 illustrates a sectional view of an embodiment of an electrical bushing according to one embodiment.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

One embodiment creates an electrical bushing for an implantable medical device, in which at least one of the disadvantages mentioned above is prevented at least in part. One embodiment provides a bushing with improved electrical properties. Features and details that are described in the context of the electrical bushing or the implantable medical device shall also apply in relation to the method, and vice versa.

One embodiment relates to an electrical bushing for use in a housing of an implantable medical device. The electrical bushing includes at least one electrically insulating base body and at least one electrical conducting element. The conducting element is set-up to establish, through the base body, at least one electrically conductive connection between an internal space of the housing and an external space. The conducting element is hermetically sealed with respect to the base body. The at least one conducting element includes at least one cermet. According to one embodiment, the at least one conducting element has a cross-section, a length L, and a resistivity rc which provide the electrically conductive connection to have an ohmic series resistance of R≦1 Ohm. R is the electrical resistance of the conducting element over the entire length of the conducting element.

According one embodiment, the ohmic series resistance R of the electrically conductive connection is R≦2 Ohm, R≦1 Ohm, R≦500 mOhm, R≦200 mOhm or R≦100 mOhm, in one embodiment, R≦50 mOhm or R≦20 mOhm or R≦10 mOhm, and in one embodiment, R≦5 mOhm, R≦2 mOhm. Depending on the current to be conducted and thus depending on the application, a range from 500 mOhm . . . 2 Ohm can be preferred in one embodiment or a resistance R of no more than 5 or 10 Ohm can be provided.

According to another embodiment, the length L of the conducting element is L≦500 μm, L≦1 m, L≦2 mm or L≦3 mm. In one embodiment, L≧300 μm or ≧400 μm, and in one embodiment ≧500 μm, ≧1 mm, ≧1.5 mm or ≧2 mm. L is the length of the conducting element over the entire longitudinal extension of the conducting element. In one embodiment, the conducting element extends along a straight line, for example, along a line perpendicular to the housing.

Moreover, one embodiment provides the area of the cross-section to be A≦15 mm², A≦10 mm², A≦5 mm², A≦2 mm², A≦1 mm², A≦0.5 mm² or A≦0.2 mm², in one embodiment A≦0.1 mm², A≦0.07 mm² or A≦0.05 mm². As another optional feature, the cross-section has a polygonal shape or a shape with a continuous curvature, for example, a rectangular, square, oval or circular shape. At least approximately circular, oval or rectangular cross-sections are preferred in one embodiment. A is the area of the cross-section of the conducting element. In one embodiment, the cross-section or area A of the cross-section is constant over the entire extension of the conducting element. Moreover, A can reflect the minimal area of the cross-section over the entire longitudinal extension of the conducting element provided the cross-section varies over the longitudinal extension.

One embodiment of the electrical bushing provides that the cermet includes a ratio of metal or alloy fraction to insulating material suited to provide the conducting element to have a resistivity of rc≦1×10³ Ohm·mm²/m, rc≦5×10² Ohm·mm²/m or rc≦1×10² Ohm·mm²/m, and in one embodiment rc≦80 Ohm·mm²/m, rc≦50 Ohm·mm²/m, rc≦20 Ohm·mm²/m, rc≦10 Ohm·mm²/m, rc≦1 Ohm·mm²/m, rc≦0.5 Ohm·mm²/m or rc≦0.3 Ohm·mm²/m. In a specific embodiment, a value R of 20 . . . 70 Ohm·mm²/m, in one embodiment, a value of approx. 50 Ohm·mm²/m, was attained in a series of experiments. The insulating material of the cermet in one embodiment is a ceramic material that can be provided as ceramic matrix. The metal or alloy fraction is in one embodiment provided by a metallic material that can be provided as metallic matrix. The parameter, rc, is the resistivity of the conducting element, whereby the letter c stands for “conductive” and reflects the property of the material of the conducting element being an electrical conductor.

Moreover, the electrical bushing according to one embodiment can include N conducting elements, whereby N≧2, N≧5, N≧10, N≧20, N≧100, N≧200, N≧500, N≧1000. The conducting elements can be arranged in one or more rows, in one embodiment along one or more straight lines. For example, the distance of consecutive conducting elements can correspond to the distance of consecutive rows. In an arrangement in multiple rows, the rows in one embodiment contain the same number of conducting elements. In a specific embodiment, the number of rows corresponds to the number of conducting elements per row, whereby the number of conducting elements per row is equal for each row. The conducting elements can be provided to be alike, for example, with regard to external dimensions, shape, and electrical properties. Moreover, the number of conducting elements can be the square of a non-negative integer larger than one. In this context, N is a non-negative integer that specifies the quantity or number of individual conducting elements per bushing.

Another embodiment provides that the conducting elements are at a distance a of a ≦1 mm, a ≦500 μm or a ≦300 μm, and in one embodiment a ≦100 μm or a ≦50 μm from each other. The distance corresponds to the distance between two closest points of two neighboring conducting elements. The distance a and a resistivity ri of an electrically insulating material of the base body of ri≧10¹² Ohm·mm²/m, ri≧10¹³ Ohm·mm²/m, ri≧10¹⁴ Ohm·mm²/m or ri≧10¹⁵ Ohm·mm²/m, and in one embodiment ri≧10¹⁶ Ohm·mm²/m, ri≧10¹⁷ Ohm·mm²/m, ri≧10¹⁸ Ohm·mm²/m or ri≧10¹⁹ Ohm·mm²/m, provide for an insulation resistance between two of the conducting elements of Ri≧10⁵, Ri≧10⁶, and in one embodiment of Ri≧10⁸ or Ri≧10⁹ Ohm. Said insulation resistance Ri is further provided by the circumferential area of the conducting elements, which corresponds to the area of a cylinder jacket of length L and a diameter value, whereby the diameter value corresponds to twice the square root of the quotient of cross-section surface area A and Pi, a mathematical constant of a circle. In other words, the diameter value corresponds to the diameter of the conducting element if the cross-section of the conducting element is circular. In this context, the parameter a is the distance between two closest points of two conducting elements arranged next to each other. The parameter, ri, is the resistivity of the material of which the base body consists. Ri is the insulation resistance of two individual conducting element that are arranged next to each other. The letter i in ri and Ri stands for “insulator”.

Moreover, one embodiment relates to an electrical bushing, whereby the at least one conducting element and the base body form a common firmly bonded boundary surface that is sufficiently tightly sealed to provide the helium leak rate to be dv≦10⁻⁷ atm·cm³/sec, dv≦10⁻⁸ atm·cm³/sec, dv≦10⁻⁹ atm·cm³/sec or dv≦10⁻¹⁰ atm·cm³/sec, and in one embodiment dv≦10⁻¹² atm·cm³/sec or dv≦10⁻¹⁵ atm—cm³/sec, whereby the leak rate is determined according to the standard, MIL-STD-883G, method 1014. The parameter, dv, relates to the volume flux through the bushing, that is, from the internal space to the external space or vice versa. In this context, the letter, “d”, stands for “differential” and the letter, “v”, stands for volume. Tightness corresponds, for example, to the definition of hermetically tight sealing, as shall be described below.

Moreover, the base body and the at least one conducting element are provided to be connected to each other in a firmly bonded manner, for example, through a firmly bonded sintered connection. Moreover, the base body and the at least one conducting element can be connected to each other through an electrically conductive soldered connection or through a glass solder connection. For example, a hard solder connection can connect the base body to the at least one conducting element in a firmly bonded manner.

And lastly, in one embodiment relates to an electrical bushing, whereby the electrical bushing includes at least one conducting element that projects from the base body and/or includes at least one conducting element having an end face that is flush with a surface of the base body. For example, an end of a conducting element can project from or be flush with the base body whereas the opposite end of the same conducting element projects or is flush.

Moreover, in one embodiment relates to an implantable medical device, for example, a cardiac pacemaker or defibrillator, that includes at least one electrical bushing according to one embodiment.

The electrical bushing according to in one embodiment is designed for use in a housing of an implantable medical device. The electrical bushing includes at least one electrically insulating base body. Moreover, the electrical bushing includes at least one electrical conducting element. The conducting element is set-up to establish, through the base body, at least one electrically conductive connection between an internal space of the housing and an external space.

In one embodiment, the electrical connection proposed in this context is an ohmic connection with low resistance—for example, for a direct current signal—, that is, a resistance R of, for example, no more than 10 Ohm, 1 Ohm, 100 mOhm, 10 mOhm or 1 mOhm. The conducting element extends through the base body, that is, along the direction of the longitudinal extension thereof. The conducting element can extend along a straight line. In one embodiment, the conducting element extends along or parallel to a longitudinal axis of the base body. The conducting element can be provided as a single part or multiple parts and can include intermediary electrical elements that provide a section of the electrically conductive connection. The conducting element can include a connecting surface that is directly adjacent to the internal space as well as a connecting surface that is directly adjacent to the external space, which serve for contacting the conducting element.

The conducting element is hermetically sealed with respect to the base body. Accordingly, conducting element and base body can include a common boundary surface. A seal is formed at the boundary surface and provides the hermetical sealing. The hermetical sealing provides the leak rate dv.

The at least one conducting element includes at least one cermet. The cermet forms a continuous structure, for example, in the longitudinal direction of the conducting element. Said structure forms at least sections of the electrically conductive connection. The cermet has a low resistivity of in one embodiment no more than 10⁶, no more than 10⁴, no more than 10³, no more than 10², and in one embodiment, no more than 10 or 1 Ohm·mm²/m. The specific conductivity is the reciprocal of the above-mentioned resistivity.

The base body is made from the insulating material either in part or fully. Said material corresponds to the at last one insulating material of the base body as described herein. The resistivity ri relates to the electrically insulating material of the base body.

Another embodiment provides the electrical bushing to include multiple conducting elements. A fraction of the conducting elements or all conducting elements extend parallel to each other. A fraction or all conducting elements of the bushing are arranged to be equidistant to each other, in one embodiment in the form of a row or in the form of multiple, equidistant rows. An electrical bushing according to one embodiment can include at least 2, 5, 10, 20, 50, 100, 200, 500 or 1000 conducting elements. The conducting elements are in one embodiment not directly electrically connected to each other. The conducting elements each form an individual electrical connection. The number of conducting elements per electrical bushing shall be denoted N.

The electrical bushing can include an electrically conductive holding element that extends around the electrical bushing.

Moreover, one embodiment relates to an implantable medical device, for example, a cardiac pacemaker or defibrillator, whereby the implantable medical device includes at least one electrical bushing according to one embodiment.

Moreover, one embodiment provides a housing for use for an implantable medical device, whereby the housing includes at least one electrical bushing according to one embodiment. Both the housing and the device include an internal space, whereby the housing and the device enclose the internal space.

One embodiment is also implemented through a use of at least one cermet-comprising conducting element in an electrical bushing for an implantable medical device. The conducting element has the longitudinal resistance R according to one embodiment.

And lastly, one embodiment is implemented through a method for the manufacture of an electrical bushing for an implantable medical device. The method includes the following steps:

a. generating at least one base body green compact for at least one base body from an electrically insulating material;

b. forming at least one cermet-containing conducting element green compact for at least one conducting element;

c. introducing the at least one conducting element green compact into the base body green compact;

d. subjecting the insulation element green compact with the at least one base body green compact to firing in order to obtain at least one base body with at least one conducting element featuring the properties described herein.

The steps a. and b. can be carried out simultaneously or in any order. Moreover, step b. can be carried out before step c. in order to form the conducting element green compact before introducing it into the base body green compact. Alternatively, step b. can be carried out during step c., whereby the cermet-containing conducting element green compact is formed while it is introduced.

For example, the manufacturing method can include additional firing steps, in which the conducting element green compact and/or the base body green compact is/are pre-sintered in order to obtain pre-sintered green compacts. Moreover, the method can provide that a holding element green compact, which surrounds the base body or the base body green compact, is provided or formed, for example, from electrically conductive or electrically insulating material.

Step a. can include a partial sintering of the base body green compact. In combination or alternatively, step b. can include a partial sintering of the conducting element green compact.

The electrically insulating material of the base body or base body green compact includes or essentially consists of the materials described above as the at least one material of the base body.

Further embodiments of the method according to one embodiment provide that a holding element green compact is produced that can, for example, be partially sintered. In one embodiment, the holding element green compact is partially sintered after forming it around the pre-sintered or non-pre-sintered base body green compact. The holding element and/or the holding element green compact includes a cermet.

In one embodiment, the electrically insulating material is one electrically insulating material or a composition of materials. The composition of materials includes at least one element from the group consisting of aluminum oxide, magnesium oxide, zirconium oxide, aluminum titanate, and piezoceramic materials.

The proposed electrical bushing is set-up for use in an implantable medical device, whereby the implantable medical device can be provided, in one embodiment, as an active implantable medical device (AIMD) and in one embodiment as a therapeutic device.

As a matter of principle, the term, implantable medical device, shall include any device which is set-up to perform at least one medical function and which can be introduced into a body tissue of a human or animal user. As a matter of principle, the medical function can include any function selected from the group consisting of a therapeutic function, a diagnostic function, and a surgical function. For example, the medical function can include at least one actuator function, in which an actuator is used to exert at least one stimulus on the body tissue, for example, an electrical stimulus.

As a matter of principle, the term, active implantable medical device—also called AIMD—shall include all implantable medical devices that can conduct electrical signals from a hermetically sealed housing to a part of the body tissue of the user and/or receive electrical signals from the part of the body tissue of the user. Accordingly, the term, active implantable medical device, includes, for example, cardiac pacemakers, cochlea implants, implantable cardioverters/defibrillators, nerve, brain, organ or muscle stimulators as well as implantable monitoring devices, hearing aids, retinal implants, muscle stimulators, implantable drug pumps, artificial hearts, bone growth stimulators, prostate implants, stomach implants or the like.

The implantable medical device, for example, the active implantable medical device, can usually include, for example, at least one housing, for example, at least one hermetically sealed housing. The housing can in one embodiment enclose at least one electronics unit, for example a triggering and/or analytical electronics unit of the implantable medical device.

In the scope of one embodiment, a housing of an implantable medical device shall be understood to be an element that encloses, at least in part, at least one functional element of the implantable medical device that is set up to perform the at least one medical function or promotes the medical function. For example, the housing includes at least one internal space that takes up the functional element fully or in part. For example, the housing can be set up to provide mechanical protection to the functional element with respect to strains occurring during operation and/or upon handling, and/or provide protection to the functional element with respect to ambient influences such as, for example, influences of a body fluid. The housing can, for example, border and/or close the implantable medical device with respect to the outside.

In this context, an internal space shall be understood herein to mean a region of the implantable medical device, for example, within the housing, which can take up the functional element fully or in part and which, in an implanted state, does not contact the body tissue and/or a body fluid. The internal space can include at least one hollow space which can be closed fully or in part. However, alternatively, the internal space can be filled up fully or in part, for example by the at least one functional element and/or by at least one filling material, for example at least one casting, for example at least one casting material in the form of an epoxy resin or a similar material.

An external space, in contrast, shall be understood to be a region outside of the housing. This can, for example, be a region which, in the implanted state, can contact the body tissue and/or a body fluid. Alternatively or in addition, the external space can just as well be or include a region that is only accessible from outside the housing without necessarily contacting the body tissue and/or the body fluid, for example a region of a connecting element of the implantable medical device that is accessible from outside to an electrical connecting element, for example an electrical plug connector.

The housing and/or, for example, the electrical bushing can, for example, be provided to be hermetically sealed such that, for example, the internal space, is hermetically sealed with respect to the external space. The hermetically sealed design envisions, for example, the tight sealing defined herein and, for example, in the claims. In this context, the term, “hermetically sealed”, can illustrate that moisture and/or gases cannot permeate through the hermetically sealed element at all or only to a minimal extent upon intended use for the common periods of time (for example 5-10 years). The leakage rate, which can be determined, for example, by leak tests, is a physical parameter that can described, for example, a permeation of gases and/or moisture through a device, for example, through the electrical bushing and/or the housing. Pertinent leak tests can be carried out with helium leak testers and/or mass spectrometers and are specified in the Mil-STD-883G Method 1014 standard. In this context, the maximal permissible helium leak rate is determined as a function of the internal volume of the device to be tested. According to the methods specified in MIL-STD-883G, method 1014, section 3.1 and taking into consideration the volumes and cavities of the devices to be tested that are used in the application of one embodiment, said maximal permissible helium leak rates can, for example, be from 1×10⁻⁸ atm*cm³/sec to 1×10⁻⁷ atm*cm³/sec. In the scope of one embodiment, the term, “hermetically sealed”, shall be understood, for example, to mean that the device to be tested (for example the housing and/or the electrical bushing and/or the housing with the electrical bushing) has a helium leak rate of less than 1×10⁻⁷ atm*cm³/sec. In one embodiment, the helium leak rate can be less than 1×10⁻⁸ atm*cm³/sec, in one embodiment, less than 1×10⁻⁹ atm*cm³/sec. For the purpose of standardization, the above-mentioned helium leak rates can also be converted into the equivalent standard air leak rate. The definition of the equivalent standard air leak rate and the conversion are specified in the ISO 3530 standard.

Electrical bushings are elements set-up to generate at least one electrically conductive path (that is, an electrically conductive connection) that extends between the internal space of the housing to at least one external point or region outside the housing, for example, situated in the external space. The electrical bushings are, for example, elements which are set-up to generate the at least one electrically conductive path based on their resistivity and structure. Accordingly, this establishes, for example, an electrical connection to leads, electrodes, and sensors that are arranged outside the housing.

Common implantable medical devices are commonly provided with a housing, which can include, on one side, a head part, also called header or connecting body, that carries connection sockets for connection of leads, also called electrode leads. The connection sockets include, for example, electrical contacts that serve to electrically connect the leads to a control electronics unit on the interior of the housing of the medical device. Usually, an electrical bushing is provided in the location, at which the electrical connection enters into the housing of the medical device, and the electrical bushing is inserted into a corresponding opening of the housing in a hermetically sealing manner.

Due to the type of use of implantable medical devices, their hermetic sealing and biocompatibility are usually amongst the foremost requirements. The implantable medical device proposed herein according to one embodiment, can be inserted, for example, into a body of a human or animal user, for example, of a patient. As a result, the implantable medical device is usually exposed to a fluid of a body tissue of the body. Accordingly, it is usually important that no body fluid penetrates into the implantable medical device and that no liquids leak from the implantable medical device. In order to ensure this, the housing of the implantable medical device, and thus the electrical bushing as well, should be as impermeable as possible, for example, with respect to body fluids.

Moreover, the electrical bushing should ensure high electrical insulation between the at least one conducting element and the housing and/or the multiple conducting elements provided that more than one conducting element are present. In this context, the insulation resistance reached in one embodiment is at least several MOhm, for example, more than 20 MOhm, and the leakage currents reached can be small, in one embodiment, less than 10 pA. Moreover, in case multiple conducting elements are present, the crosstalk and electromagnetic coupling between the individual conducting elements in one embodiment are below the specified thresholds for medical applications. Said insulation resistances correspond to the insulation resistance Ri.

The electrical bushing disclosed according to one embodiment is well-suited for the above-mentioned applications. Moreover, the electrical bushing can also be used in other applications that are associated with special requirements with regard to biocompatibility, tight sealing, and stability.

The electrical bushing according to one embodiment can meet, for example, the above-mentioned tight sealing requirements and/or the above-mentioned insulation requirements.

As mentioned above, the electrical bushing includes at least one electrically insulating base body. In the scope of one embodiment, a base body shall be understood to mean an element that serves a mechanical holding function in the electrical bushing, for example in that the base body holds or carries the at least one conducting element either directly or indirectly. For example, the at least one conducting element can be embedded in the base body directly or indirectly, fully or partly, for example, through a firmly bonded connection between the base body and the conducting element and in one embodiment through co-sintering of the base body and the conducting element. For example, the base body can have at least one side facing the internal space and at least one side facing the external space and/or accessible from the external space.

As mentioned above, the base body is provided to be electrically insulating. This means that the base body, fully or at least regions thereof, is made from at least one electrically insulating material. In this context, an electrically insulating material shall be understood to mean a material with a resistivity of at least 10⁷ Ohm*m, in one embodiment, of at least 10⁸ Ohm*m, in one embodiment of at least 10⁹ Ohm*m, and in one embodiment of at least 10¹¹ Ohm*m. Said resistivity in one embodiment corresponds to the resistivity ri of the electrically insulating material of the base body. For example, the base body can be provided such that, as mentioned above, a flow of current between the conducting element and the housing and/or between multiple conducting elements is at least largely prevented, for example through the resistivity values between the conducting element and the housing as specified above being implemented. For example, the base body can include at least one ceramic material.

In this context, a conducting element or electrical conducting element shall generally be understood to mean an element set-up to establish an electrical connection between at least two sites and/or at least two elements. For example, the conducting element can include one or more electrical conductors, for example metallic conductors. In the scope of one embodiment, the conducting element is made fully or partly of at least one cermet, as mentioned above. In addition, one or more other electrical conductors, for example metallic conductors, can be provided. The conducting element can, for example, be provided in the form of one or more contact pins and/or curved conductors. Moreover, the conducting element can include, for example, on a side of the base body and/or electrical bushing facing the internal space or on a side of the base body and/or electrical bushing facing the external space or accessible from the external space, one or more connecting contacts, for example one or more plug-in connectors, for example one or more connecting contacts, which project from the base body or can be electrically contacted through other means from the internal space and/or the external space.

The at least one conducting element can establish the electrically conducting connection between the internal space and the external space in a variety of ways. For example, the conducting element can extend from at least one section of the conducting element that is arranged on the side of the base body facing the internal space to at least one section of the conducting element arranged on the side facing the external space or accessible from the external space. However, other arrangements are also feasible as a matter of principle. Accordingly, the conducting element can just as well include a plurality of partial conducting elements that are connected to each other in an electrically conducting manner. Moreover, the conducting element can extend into the internal space and/or the external space. For example, the conducting element can include at least one region that is arranged in the internal space and/or at least one region that is arranged in the external space, whereby the regions can, for example, be electrically connected to each other. Various exemplary embodiments shall be illustrated in more detail below.

The at least one conducting element can include, on a side of the base body and/or electrical bushing facing the internal space or on a side of the base body and/or electrical bushing facing the external space or accessible from the external space, at least one electrical connecting element and/or be connected to an electrical connecting element of this type. For example, as described above, one or more plug-in connectors and/or one or more contact surfaces and/or one or more contact springs and/or one or more types of electrical connecting elements can be provided on one or both of said sides. The at least one optional connecting element can, for example, be a component of the at least one conducting element and/or can be connected to the at least one conducting element in an electrically conducting manner For example, one or more conducting elements of the bushing can be contacted to one or more internal connecting elements and/or one or more external connecting elements. The material of the internal connecting elements should be suited for permanent connection to the conducting element. The external connecting elements should be biocompatible and should be such that they can be permanently connected to the at least one conducting element.

The electrically insulating base body can support, as a bearing, for example, the at least one conducting element. The at least one material of the base body, that is, the electrically insulating material of the base body, should in one embodiment be biocompatible, as illustrated above, and should have sufficiently high insulation resistance. It has proven to be advantageous in one embodiment for the base body to include one or more materials selected from the group consisting of: aluminum oxide (Al₂O₃), zirconium dioxide (ZrO₂), aluminum oxide-toughened zirconium oxide (ZTA), zirconium oxide-toughened aluminum oxide (ZTA—Zirconia Toughened Aluminum—Al₂O₃/ZrO₂), yttrium-toughened zirconium oxide (Y-TZP), aluminum nitride (AlN), magnesium oxide (MgO), piezoceramic materials, barium (Zr, Ti) oxide, barium (CE, Ti) oxide, and sodium-potassium-niobate. The materials are also called materials and, for example, can be provided as compositions of materials.

An edge body, also called holding element, reaches around the base body and serves as connecting element to the housing of the implantable device. The materials of the edge body must be biocompatible, easy to process, corrosion-resistant, and permanently connectable to the base body and the housing in a firmly bonded manner. It has proven to be advantageous in one embodiment for the edge body according to one embodiment to include at least one of the following metals and/or an alloy based on at least one of the following metals: platinum, iridium, niobium, molybdenum, tantalum, tungsten, titanium, cobalt-chromium alloys or zirconium. Alternatively, the edge body can include a cermet, whereby a cermet is also advantageous in one embodiment with regard to tight sealing and manufacturing method.

In the proposed electrical bushing, the at least one conducting element includes at least one cermet.

The base body can, for example, be made fully or partly from one or more sinterable materials, for example, from one or more ceramic-based sinterable materials. The conducting element or elements can fully or partly be made of one or more cermet-based sinterable materials. Moreover, the at least one conducting element can also, as mentioned above, include one or more additional conductors, for example one or more metallic conductors.

In the scope of one embodiment, “cermet” shall refer to a composite material made of one or more ceramic materials in at least one metallic matrix or a composite material made of one or more metallic materials in at least one ceramic matrix. For production of a cermet, for example, a mixture of at least one ceramic powder and at least one metallic powder can be used to which, for example, at least one binding agent and, if applicable, at least one solvent can be added. The ceramic powder or powders of the cermet in one embodiment have a mean grain size of less than 10 μm, in one embodiment less than 5 μm, and in one embodiment less than 3 μm. The metallic powder or powders of the cermet in one embodiment have a mean grain size of less than 15 μm, in one embodiment less than 10 μm, and in one embodiment less than 5 μm. For production of a base body, for example, at least one ceramic powder can be used to which, for example, at least one binding agent and, if applicable, at least one solvent can be added. In this context, the ceramic powder or powders in one embodiment has/have a mean grain size of less than 10 μm (1 μm are equal to 1×10⁻⁶ m), in one embodiment less than 5 μm, in one embodiment less than 3 μm. For example, the median value or the d50 value of the grain size distribution is considered to be the mean grain size in this context. The d50 value corresponds to the value at which 50 percent of the grains of the ceramic powder and/or metallic powder are finer and 50% are coarser than the d50 value.

In the scope of the one embodiment, sintering or a sintering process shall generally be understood to mean a method for producing materials or work-pieces, in which powdered, for example, fine-grained, ceramic and/or metallic substances are heated and thus connected. This process can proceed without applying external pressure onto the substance to be heated or can, for example, proceed under elevated pressure onto the substance to be heated, for example under a pressure of at least 2 bar, in one embodiment higher pressures, for example pressures of at least 10 bar, for example, at least 100 bar, or even at least 1000 bar. The process can proceed, for example, fully, or partly at temperatures below the melting temperature of the powdered material, for example at temperatures of 700° C. to 1400° C. The process can be implemented, for example, fully, or partly in a tool and/or a mould such that a forming step can be associated with the sintering process. Aside from the powdered materials, a starting material for the sintering process can include at least one further material, for example one or more binding agents and/or one or more solvents. The sintering process can proceed in one or more steps, whereby additional steps can precede the sintering process, for example one or more forming steps and/or one or more debinding steps.

A method can be used, for example, in the manufacture of the at least one conducting element and/or optionally in the manufacture of the at least one base body, in which at least one green compact is manufactured first, subsequently at least one brown compact is manufactured from said green compact, and subsequently the finished work-piece is manufactured from said brown compact through at least one sintering step. In this context, separate green compacts and/or separate brown compacts can be manufactured for the conducting element and the base body and can be connected subsequently. Alternatively, one or more common green compacts and/or brown compacts can be produced for the base body and the conducting element. Alternatively again, separate green compacts can be produced first, said green compacts can then be connected, and subsequently a common brown compact can be produced from the connected green compact. In general, a green compact shall be understood to mean a preform body of a work-piece which includes the starting material, for example the at least one ceramic and/or metallic powder, as well as, if applicable, the one or more binding agents and/or one or more solvents. A brown compact shall be understood to mean a pre-form body which is generated from the green compact through at least one debinding step, for example at least one thermal and/or chemical debinding step, whereby the at least one binding agent and/or the at least one solvent is/are removed, at least partly, from the pre-form body in the debinding step.

The sintering process, for example, of a cermet, but of the base body just as well, for example, can proceed comparable to a sintering process that is commonly used for homogeneous powders. For example, the material can be compacted in the sintering process at high temperature and, if applicable, high pressure such that the cermet is virtually sealed tight or has no more than closed porosity. Usually, cermets are characterized by their particularly high toughness and wear resistance. Compared to sintered hard metals, a cermet-containing transmission element usually has a higher thermal shock and oxidation resistance and usually a thermal expansion coefficient that is matched to a surrounding insulator.

For the bushing according to one embodiment, the at least one ceramic component of the cermet can include, for example, at least one of the following materials: aluminum oxide (Al₂O₃), zirconium dioxide (ZrO₂), aluminum oxide-toughened zirconium oxide (ZTA), zirconium oxide-toughened aluminum oxide (ZTA—Zirconia Toughened Aluminum—Al₂O₃/ZrO₂), yttrium-toughened zirconium oxide (Y-TZP), aluminum nitride (AlN), magnesium oxide (MgO), piezoceramic materials, barium (Zr, Ti) oxide, barium (CE, Ti) oxide, or sodium-potassium-niobate.

For the bushing according to one embodiment, the at least one metallic component of the cermet can include, for example, at least one of the following metals and/or an alloy based on at least one of the following metals: platinum, iridium, niobium, molybdenum, tantalum, tungsten, titanium, cobalt or zirconium. An electrically conductive connection is usually established in the cermet when the metal content exceeds the so-called percolation threshold at which the metal particles in the sintered cermet are connected to each other, at least in spots, such that electrical conduction is enabled. For this purpose, experience tells that the metal content should be 25% by volume and more, in one embodiment 32% by volume, in one embodiment, more than 38% by volume, depending on the selection of materials.

In the scope of one embodiment, the terms, “including a cermet,” “cermet-including,” “comprising a cermet,” and “cermet-containing”, are used synonymously. Accordingly, the terms refer to the property of an element, being that the element contains cermet. This meaning also includes the variant of an embodiment in that elements, for example the conducting element, consist of a cermet, that is, are fully made of a cermet.

In one embodiment, both the at least one conducting element and the base body can include one or more components which are or can be manufactured in a sintering procedure, or the at least one conducting element and the base body are or can both be manufactured in a sintering procedure. For example, the base body and the conducting element are or can be manufactured in a co-sintering procedure, that is, a procedure of simultaneous sintering of these elements. For example, the conducting element and the base body each can include one or more ceramic components that are manufactured, and in one embodiment compacted, in the scope of at least one sintering procedure.

For example, a base body green compact can be manufactured from an insulating composition of materials. This can proceed, for example, by compressing the composition of materials in a mould. In this context, the insulating composition of materials is a powder mass in one embodiment, in which the powder particles illustrate at least minimal cohesion. In this context, the production of a green compact proceeds, for example, through compressing powder masses or through forming by plastic shaping or casting and subsequent drying.

Said procedural steps can also be utilized to form at least one cermet-containing conducting element green compact. In this context, one embodiment can provide that the powder, which is compressed to form the conducting element green compact, is cermet-containing or consists of a cermet or includes at least one starting material for a cermet. Subsequently, the two green compacts—the base body green compact and the conducting element green compact—can be combined. The production of the conducting element green compact and the base body green compact can just as well proceed simultaneously, for example, by multi-component injection molding, co-extrusion, etc., such that there is no longer a need to connect them subsequently.

While the green compacts are being sintered, they are in one embodiment subjected to a heat treatment below the melting temperature of the powder particles of the green compact. This usually leads to compaction of the material and ensuing substantial reduction of the porosity and volume of the green compacts. Accordingly, in one embodiment of the method the base body and the conducting element can be sintered jointly. Accordingly, there is in one embodiment no longer a need to connect the two elements subsequently.

Through the sintering, the conducting element becomes connected to the base body in one embodiment in a positive fit-type and/or non-positive fit-type and/or firmly bonded manner. This achieves hermetic integration of the conducting element into the base body in one embodiment. In one embodiment, there is no longer a need for subsequent soldering or welding of the conducting element into the base body. Rather, a hermetically sealing connection between the base body and the conducting element is attained through the joint sintering in one embodiment and utilization of a cermet-containing green compact in one embodiment.

One refinement of the method according to one embodiment is characterized in that the sintering includes only partial sintering of the at least one optional base body green compact, whereby said partial sintering can effect and/or include, for example, the debinding step mentioned above. In one embodiment, the green compact is heat-treated in the scope of said partial sintering. This is usually already associated with some shrinkage of the volume of the green compact. However, the volume of the green compact has not yet reached its final state. Rather, another heat treatment is usually needed—a final sintering—in which the green compact(s) is/are shrunk to its/their final size. In the scope of said variant of an embodiment, the green compact is in one embodiment sintered only partly in order to attain a certain stability to render the green compact easier to handle.

The starting material used for producing at least one conducting element green compact and/or at least one base body green compact can, for example, be a dry powder or include a dry powder, whereby the dry powder is compressed in the dry state into a green compact and illustrates sufficient adhesion to maintain its compressed green compact shape. However, optionally, the starting material can include one or more further components in addition to the at least one powder, for example, as mentioned above, one or more binding agents and/or one or more solvents. Said binding agents and/or solvents, for example organic and/or inorganic binding agents and/or solvents, are generally known to the person skilled in the art, and are commercially available, for example. The starting material can, for example, include one or more slurries or be a slurry. In the scope of one embodiment, a slurry is a suspension of particles of a powder made of one or more materials in a liquid binding agent, and, if applicable, in a water-based or organic binding agent. A slurry has a high viscosity and can easily be formed into a green compact without the application of high pressure, for example through casting or injection molding or plastic forming

In the case of green compacts made from slurries, the sintering process, which is generally carried out below the melting temperature of the ceramic, cermet or metal materials that are used, but in individual cases can also be carried out just above the melting temperature of the lower melting component of a multi-component mixture, this usually being the metal component, leads to the binding agent slowly diffusing from the slurry. Overly rapid heating leads to a rapid increase of the volume of the binding agent by transition to the gas phase and destruction of the green compact or formation of undesired defects in the work-piece.

Thermoplastic and duroplastic polymers, waxes, thermogelling substances and/or surface-active substances, for example, can be used as binding agent—also called binder. In this context, these can be used alone or as binding agent mixtures of multiple components of this type. If individual elements or all elements of the bushing (base body green compact, conducting element green compact, bushing blank) are produced in the scope of an extrusion procedure, the composition of the binding agent should be such that the line of the elements extruded through the nozzle is sufficiently stable in shape for the shape defined by the nozzle to easily be maintained. Suitable binders, also called binding agents, are known to the person skilled in the art.

In contrast to one embodiment, according to which a conducting element includes at least one cermet, the prior art has a metallic wire or other metallic work-piece be the conducting element. A conducting element, which, according to one embodiment, is provided with a cermet, can be connected to the base body easily, since the cermet and the insulation element are or include ceramic substances and/or a ceramic material. The base body can also be called insulation element in order to address the electrical function; in this context, the two terms are exchangeable. Green compacts of both the conducting element and the base body can be produced and subsequently subjected to a sintering process. The resulting electrical bushing is not only particularly biocompatible and durable, but also possesses good hermetic sealing properties. Thus, no fissures or connecting sites still to be soldered result between the conducting element and the base body. Rather, sintering results in the base body and the conducting element becoming connected. One variant of an embodiment therefore provides the at least one conducting element to consist of a cermet. In this variant of an embodiment, the conducting element includes not only components made of cermet, but is fully made of a cermet. Generally, cermets are characterized by their particularly high toughness and wear resistance. The “cermets” and/or “cermet-containing” substances can, for example, be or include cutting materials related to hard metals which can dispense with tungsten carbide as the hard substance and can be produced, for example, by a powder metallurgical route. A sintering process for cermets and/or the cermet-containing conducting element proceeds, for example, alike a process for homogeneous powders except that, at identical compression force, the metal is usually compacted more strongly than the ceramic material. Compared to sintered hard metals, the cermet-containing conducting element usually illustrates higher resistance to thermal shock and oxidation. As mentioned above, the ceramic components can be, for example, aluminum oxide (Al₂O₃) and/or zirconium dioxide (ZrO₂), whereas for example, niobium, molybdenum, titanium, cobalt, zirconium, chromium are conceivable as metallic components.

For integration of the electrical bushing into the housing of a cardiac pacemaker, the electrical bushing can include a holding element. Said holding element is arranged about the base body in a wreath-like arrangement. The term, wreath-like, is used to refer to a sleeve shape with a rim that extends radially outward. The holding element surrounds the base body, in one embodiment along its entire circumference. The purpose of the holding element is to establish a non-positive fit- and/or positive fit-type connection to the housing. A fluid-tight connection between the holding element and the housing must be established in the process. In one embodiment, the electrical bushing includes a holding element that includes a cermet. The cermet-containing holding element can be connected to the housing of the implantable medical device in an easy, durable and hermetically sealed manner. Another embodiment provides the holding element to not only include a cermet, but to consist of a cermet.

Moreover, it is conceivable that the conducting element and the holding element are made from the same material. In this variant, the same materials are used for both the conducting element and the holding element. This relates, for example, to a durable, conductive, and biocompatible cermet. Since both the holding element and the conducting element are still to be connected to metallic components, both must include means to be welded or soldered to them. If a cermet is found that meets the pre-requisites specified above, said cermet can be used for both the holding element and the conducting element in order to obtain a particularly inexpensive electrical bushing.

In electrical terms, the base body can also be considered to be an insulation element that is electrically insulating. The base body is made from an electrically insulating material, in one embodiment from an electrically insulating composition of materials. The base body is set-up to electrically insulate the conducting element from the holding element or—(in case no holding element is provided)—from the housing and/or other objects of the implantable medical device. Electrical signals that are propagated through the conducting wire shall not be attenuated or short-circuited by contacting the housing of the implantable device. In addition, the composition of the base body must be biocompatible for implantation in medical applications. For this reason, it is preferred in one embodiment that the base body consists of a glass-ceramic or glass-like material. It has been found to be preferred in one embodiment that the insulating composition of materials of the base body is at least any one from the group, aluminum oxide (Al₂O₃), magnesium oxide (MgO), zirconium oxide (ZrO₂), aluminum titanate (Al₂TiO₅), and piezoceramic materials. In this context, aluminum oxide features high electrical resistance and low dielectric losses. These properties are supplemented by the additional high thermal resistance and good biocompatibility.

Another refinement of the bushing according to one embodiment is characterized in that the holding element includes at least one flange, whereby the flange, for example, is conductive like a metal. The purpose of the flange is to seal the electrical bushing with respect to a housing of the implantable device. The holding element holds the electrical bushing in the implantable device. In the variant of an embodiment described herein, the holding element includes at least one flange on an external side. These flanges form a bearing, which can be engaged by the lids of the implantable medical device, for example, engaged in a tightly sealing manner Accordingly, the holding element including the flanges connected to it can have a U- or H-shaped cross-section. Integrating at least one flange into the holding element ensures that the electrical bushing is integrated into the implantable device in a safe, impact-resistant and durable manner. In addition, the flanges can be provided such that the lids of the implantable device are connected clip-like to the holding element in a non-positive fit-type or positive fit-type manner.

Another refinement of the electrical bushing according to one embodiment is characterized in that the at least one flange includes a cermet. In the scope of said variant of an embodiment, both the holding element and the flange include a cermet. Both the flange and the holding element are made of the same material in one embodiment. By providing the flange as a cermet, the flange can be sintered easily and inexpensively jointly with the insulation element and the conducting element as part of the holding element in the scope of the method described here.

One embodiment also includes a use of at least one cermet-comprising conducting element in an electrical bushing for an implantable medical device. Features and details that were described in the context of the electrical bushing and/or the method shall obviously also apply in relation to the use of a cermet-containing conducting element.

The scope of one embodiment also includes an implantable medical device, for example, a cardiac pacemaker or defibrillator, having an electrical bushing according to at least one of the preceding claims. Features and details that were described in the context of the electrical bushing and/or the method shall obviously also apply in relation to the implantable medical device.

Features and properties that are described in the context of the electrical bushing shall also apply in relation to the method according to one embodiment, and vice versa.

The method according to one embodiment provides both the base body and the conducting element to include ceramic components that are processed in the scope of a sintering process. In the scope of step a), a base body green compact is generated from an insulating composition of materials. This can be done by compressing the composition of materials in a mould. In this context, the insulating composition of materials is a powder mass in one embodiment, in which the powder particles illustrate at least minimal cohesion. Commonly, this is realized in that a grain size of the powder particles does not exceed 0.5 mm, whereby a mean grain size of less than 10 μm is used in one embodiment. It is preferable to use grain sizes described above in one embodiment. In this context, the manufacture of the green compact proceeds either by compressing powder masses or by forming and subsequent drying. Said procedural steps are also utilized to form the cermet-containing conducting element green compact. In this context one embodiment provides the powder, which is compressed into the conducting element green compact, to be cermet-containing or to consist of a cermet. The green compacts—for example, the base body green compact and the conducting element green compact—are in one embodiment combined subsequent to this step. After this step, which is called step c), the two green compacts are subjected to firing—which is also called sintering. In the process of sintering or firing, the green compacts are subjected to a heat treatment below the melting temperature of the powder particles of the green compact. This leads to a substantial reduction of the porosity and volume of the green compacts. The special feature according to one embodiment of the method is therefore that the base body and the conducting element are jointly subjected to firing and the conducting element is generated to have at least one conductive surface. Subsequently, there is no longer a need to connect the two elements and, for example, there is no need to generate a conductive surface in an additional step. Through the firing process, the conducting element becomes connected to the base body in a positive fit-type and/or non-positive fit-type and/or firmly bonded manner. This achieves hermetic integration of the conducting element into the base body. There is no longer a need for subsequent soldering or welding of the conducting element into the base body. Rather, through the joint firing and the utilization of a cermet-containing green compact, that is, of the conducting element green compact, a hermetically sealing connection between the base body and the conducting element is attained.

A refinement of the method according to one embodiment is characterized in that step a) includes a partial sintering of the base body green compact. The green compact of the insulation element is heat-treated in the scope of said partial sintering. This is already associated with some shrinkage of the volume of the insulation element green compact. However, the volume of the green compact does not reach its final state. Rather, this requires another heat treatment in the scope of step d), in which the base body green compact with the conducting element green compact are shrunk to their final size. In the scope of said variant of an embodiment, the green compact is heat treated only partly in order to already attain a certain surface hardness to render the base body green compact easier to handle. This is expedient for example, in the case of insulating compositions of materials which can be compressed into a green compact shape only with some difficulty.

For example, a component of the bushing according to one embodiment is called green compact unless all sintering steps have been carried out. Accordingly, even a pre-sintered or partly sintered or heat-treated green compact is called green compact until all heat treatment or sintering steps have been completed.

Another variant of the embodiment is characterized in that the conducting element green compact is also already partly sintered in step b). As described above for the base body green compact, the conducting element green compact can also be partly sintered in order to already attain a certain surface stability. It needs to be noted in this context that the final complete sintering occurs no earlier than in step d). Accordingly, the conducting element green compact attains its final size only in step d).

Another refinement of the method is characterized in that at least one cermet-containing holding element green compact for a holding element is generated. The conducting element green compact is introduced into the base body green compact. The base body green compact is introduced into the holding element green compact. The base body green compact is subjected to firing jointly with the at least one conducting element green compact and the holding element green compact. This results in a base body with a conducting element and a holding element.

The special feature of this procedural step is that, not only the conducting element green compact and the base body green compact, but also the holding element green compact is sintered in one step. All three green compacts are generated, then joined, and subsequently subjected to firing or sintering as a unit. In a particular variant of an embodiment, producing the at least one cermet-containing holding element green compact can include a partial sintering. As before, one embodiment provides the fringe green compact to be partly sintered in order to attain higher surface stability. In this context, the base body green compact can thus form the dielectric layer or a piezoelectric body for the filter structure or a receptacle for a frequency-selective component.

A specific exemplary embodiment of a method for the manufacture of a bushing according to one embodiment is presented in the following.

In the first step, a cermet mass is produced from platinum (Pt) and aluminum oxide (Al₂O₃) containing 10% zirconium dioxide (ZrO₂). The following starting materials are used for this purpose:
40 vol. % Pt powder with a mean grain size of 10 μm, and
60 vol. % Al₂O₃/ZrO₂ powder with a relative ZrO₂ content of 10% and a mean grain size of 1 μm.

The two components were mixed, water and a binding agent were added, and the sample was homogenized through a kneading process. Analogous to the first step, a ceramic mass is produced in a second step from a powder with an Al₂O₃ content of 90% and a ZrO₂ content of 10%. The mean grain size was approx. 1 μm. As before, water and a binding agent were added to the ceramic powder and the sample was homogenised. In a third step, the ceramic mass made of aluminium oxide with a 10% zirconium dioxide content produced in step two was converted to a shape of a base body. Made from the cermet mass produced in the first step, a cermet body that contained a mixture of platinum powder and aluminium oxide with a zirconium dioxide content of 10%, was introduced as green compact into an opening in the base body green compact. Subsequently, the ceramic mass was compacted in the mould. Then the cermet and the ceramic component were subjected to debinding at 500° C. and the sintering was finished at 1650° C.

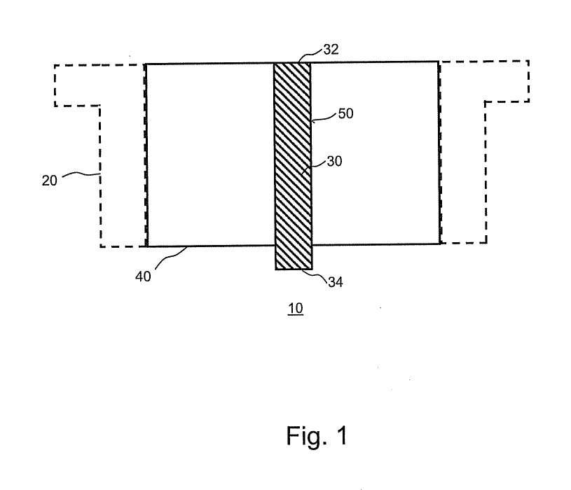

FIG. 1 illustrates a sectional view of an embodiment of the electrical bushing 10 according to one embodiment. The electrical bushing 10 illustrated in FIG. 1 is radially surrounded by an optional holding element 20 that is indicated by dashed lines. The optional holding element 20 is made from a conductive material, for example, from a cermet, and includes a circumferential rim in order to simplify the insertion into a housing (not illustrated). Alternatively, the holding element 20 can just as well be provided to be made from metal or a metal alloy.

The electrical bushing 10 includes a conducting element 30 and a base body 40, whereby the base body is electrically insulating and the conducting element is electrically conductive. The conducting element 30 extends fully through the base body 40 and thus provides an electrically conductive connection between an internal space and an external space. In FIG. 1, the external space is arranged above the electrical bushing 10 and the internal space is arranged below the electrical bushing 10. In one embodiment, the internal space and/or external space are directly adjoining to the bushing 10 illustrated in the figures.

The conducting element 30 extends along a straight line. Said line corresponds to the longitudinal axis of the bushing 10. The cross-section of the conducting element 30 is circular. This results in a circular cylinder shape, whereby the end faces 32 and 34 of the circular cylinder shape serve for contacting and the section of the cylinder jacket surrounded by the base body 40 and the adjacent base body adjacent to it form a boundary surface 50. One section of the conducting element 30 projects from the base body 40 and is not surrounded by the base body. Said section of the conducting element projects into the adjacent space below the bushing 10 and right next to the base body.

End faces 32 and 34 of the conducting element 30 are directly adjacent to the space that is adjacent to the upper side and/or the underside of the bushing. The end faces of the conducting element 30 can be flush on one side of the bushing 10, and can project from one of the sides of the bushing 10. The end face 32 of the conducting element 30 is flush with the upper side of the bushing 10 and thus is flush with the upper side. The end face 34 of the conducting element is an end face of the conducting element 30 that projects from the base body. One of the ends of the conducting element 30 thus projects from the base body and forms an end face 34 which is offset outwards with respect to the base body. This enables simplified contacting—depending on the contacting structure.

The base body 40 surrounds the conducting element 30 around its entire circumference. The base body 40 and the conducting element 30 contact each other directly, whereby the resulting boundary surface 50 equally reflects the contour of the inside of the base body 40 and the circumferential contour of the conducting element 30. The base body 40 and the conducting element 30 are connected in a firmly bonded manner, for example, through joint sintering, at the boundary surface 50.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

1. An electrical bushing for use in a housing of an implantable medical device,
whereby the electrical bushing comprises at least one electrically insulating base body and at least one electrical conducting element;
whereby the conducting element is set-up to establish, though the base body, at least one electrically conductive connection between an internal space of the housing and an external space;
whereby the conducting element is hermetically sealed with respect to the base body; and
whereby the at least one conducting element comprises at least one cermet;
characterized in that the at least one conducting element has a cross-section, a length L, and a resistivity rc which provide the electrically conductive connection to have an ohmic series resistance of R≦2 Ohm.

2. The electrical bushing according to claim 1, whereby R≦100 mOhm.

3. The electrical bushing according to claim 1, whereby R≦2 mOhm.

4. The electrical bushing according to claim 1, whereby L≦500 μm.

5. The electrical bushing according to claim 1, whereby L≧500 μm.

6. The electrical bushing according to claim 1, whereby L≧2 mm.

7. The electrical bushing according to claim 1, whereby the cross-section has an area A of the cross section and A is A≦15 mm².

8. The electrical bushing according to claim 1, whereby the cross-section has an area A of the cross section and A is A≦0.05 mm².

9. The electrical bushing according to claim 1, whereby the cross-section has a polygonal shape or a shape with a continuous curvature comprising one of a group comprising a rectangular, square, oval and circular shape.

10. The electrical bushing according to claim 1, whereby the cermet comprises a ratio of metal or alloy fraction to insulating material suited to provide the conducting element to have a resistivity of rc≦1×10³ Ohm·mm²/m.

11. The electrical bushing according to claim 1, whereby the cermet comprises a ratio of metal or alloy fraction to insulating material suited to provide the conducting element to have a resistivity of rc≦0.3 Ohm·mm²/m.

12. The electrical bushing according to claim 1, whereby the bushing comprises N conducting elements, whereby N≧2.

13. The electrical bushing according to claim 1, whereby the bushing comprises N conducting elements, whereby N≧1000.

14. The electrical bushing according to claim 13, whereby the conducting elements are at a distance a of a≦1 mm, and the distance a resistivity ri of an electrically insulating material of the base body of ri≧10¹² Ohm·mm²/m, provide for an insulation resistance between two of the conducting elements of Ri≧10⁵ Ohm.

15. The electrical bushing according to claim 13, whereby the conducting elements are at a distance a of a ≦50 μm, and the distance a resistivity ri of an electrically insulating material of the base body of ri≧10¹⁹ Ohm·mm²/m, provide for an insulation resistance between two of the conducting elements of Ri≧10⁹ Ohm.

16. The electrical bushing according to claim 1, whereby the at least one conducting element and the base body form a common firmly bonded boundary surface that is sufficiently tightly sealed to provide the helium leak rate to be dv≦10⁻⁷ atm·cm³/sec, whereby the leak rate is determined according to the standard, MIL-STD-883G, method 1014.

17. The electrical bushing according to claim 1, whereby the at least one conducting element and the base body form a common firmly bonded boundary surface that is sufficiently tightly sealed to provide the helium leak rate to be dv≦10⁻¹⁵ atm·cm³/sec, whereby the leak rate is determined according to the standard, MIL-STD-883G, method 1014.

18. The electrical bushing according to claim 1, whereby the electrical bushing comprises at least one conducting element that projects from the base body and/or comprises at least one conducting element having an end face that is flush with a surface of the base body.

19. An implantable medical device comprising:
a housing; and
an electrical bushing used in the housing and comprising at least one electrically insulating base body and at least one electrical conducting element;
whereby the conducting element is set-up to establish, though the base body, at least one electrically conductive connection between an internal space of the housing and an external space;
whereby the conducting element is hermetically sealed with respect to the base body; and
whereby the at least one conducting element comprises at least one cermet;
characterized in that the at least one conducting element has a cross-section, a length L, and a resistivity rc which provide the electrically conductive connection to have an ohmic series resistance of R≦2 Ohm.

20. The implantable medical device of claim 19, whereby the implantable medical device comprises a cardiac pacemaker or defibrillator.
